(19) United States
(12) Patent Application Publication
(10) Pub. No.: US 2012/0245443 A1
(43) Pub. Date: Sep. 27, 2012

(54) BIOLOGICAL LIGHT MEASUREMENT DEVICE

(76) Inventors: Hirokazu Atsumori, Kawagoe (JP); Hiroki Sato, Shiki (JP); Masashi Kiguchi, Kawagoe (JP)

(21) Appl. No.: 13/512,020

(22) PCT Filed: Nov. 11, 2010
(86) PCT No.: PCT/JP2010/070116

§ 371 Date: May 24, 2012

(30) Foreign Application Priority Data
Nov. 27, 2009 (JP) 2009-269435

(51) Int. Cl. A61B 5/1455

(52) U.S. Cl. 600/328

(57) ABSTRACT

The mental state, such as mood or emotion, of an individual can be apprehended by a method using non-invasive biological light measurement technology. A biological light measurement device, which has an irradiation section, presents different tasks (at least a first task and a second task) to a subject, hemoglobin signals based on changes in the concentration of oxygenated hemoglobin and deoxygenated hemoglobin in the subject are calculated from the strength of light detected by a detection section, and a relative value using the hemoglobin signal at a predetermined measurement channel with respect to the first task, and the hemoglobin signal at a different predetermined measurement channel with respect to the second task is calculated.

### TECHNICAL FIELD

The present invention relates to a biological light measurement device for measuring, using light, information inside a living body, a change in concentration of light-absorbing material in particular, and more particularly, to a biological light measurement device which provides information for supporting brain activity assessment based on data measured by the biological light measurement device.

### BACKGROUND ART

Devices which can measure information inside a living body in a simple manner without harming the living body are used in such fields as clinical treatment and brain science. Among the measurement methods used by such devices, measurement by use of light is very effective. The first reason why is that the oxygen metabolism function inside a living body corresponds to the concentrations of specific pigments (such as hemoglobin, cytochrome aa3 and myoglobin) in the living body and the concentrations of such pigments can be known by measuring amounts of light absorption. The second and third reasons why light measurement is effective are that light can be handled in a simple manner using optical fibers and that light used in compliance with safety standards is harmless to living bodies.

A biological light measurement device which, making use of the above advantages of light measurement, measures the interior of a living body using plural light beams ranging in wavelength from visible light wavelengths to infrared light wavelengths and two-dimensionally displays the result of measurement is disclosed, for example, in the patent document 1. In the biological light measurement device disclosed in the patent document 1: light beams are generated using semiconductor lasers; the light beams generated are irradiated to plural parts of a subject; light beams transmitted through or reflected from the living body are detected at plural locations; the light beams detected are led to photodiodes through optical fibers; and living body information related with, for example, blood circulation, hemodynamic status and hemoglobin concentration changes is obtained based on the amounts of detected light beams; and the living body information obtained is two-dimensionally displayed.

The above technique is expected to find applications for assessing individuals' everyday mental states, for example, about mood or emotion. This is because, whereas the functional magnetic resonance imaging (fMRI) among the related-art techniques requires measurement to be performed in a very noisy environment with a subject restrained, the biological light measurement technique compared with the fMRI technique has an advantage that measurement can be performed in a simple manner in an everyday environment. Individuals' mood and emotion, in particular, is difficult to objectively grasp. If objective assessment of individuals' mental states is enabled by biological light measurement, biological light measurement will find, taking advantage of its measurement simplicity, applications such as mental health check and sensitivity assessment to be performed under everyday circumstances. It has, however, been impossible to assess the mental state of an individual based on brain activity signals obtained by biological light measurement.

### CITATION LIST

### Patent Literature

Patent document 1: Japanese Patent Laid-Open No. Hei 9 (1997)-98972

### SUMMARY OF THE INVENTION

### Technical Problem

The biological light measurement technique that visualizes the state of brain activity is expected to find applications for providing information about individuals' mental states, for example, about mood or emotion. The related-art fMRI technique requiring a subject to be restrained and involving large noise cannot avoid imposing an extraordinary environment and measurement conditions on the subject. There has not been any method in which an individual's mental state, for example, about mood or emotion can be grasped using a biological light measurement technique applicable in an everyday environment.

The present invention provides a biological light measurement device which can assess an individual's mental state, for example, about mood or emotion, in an everyday environment.

To solve the above problem, the present invention provides a biological light measurement device including: one or multiple irradiation means for emitting light to a subject; one or multiple detection means for detecting light transmitted through or reflected from a subject; multiple measurement channels including multiple combinations of the irradiation means and the detection means; a task presentation section which at least presents multiple different tasks (a first task and a second task) to a subject; a computing section which calculates, based on intensities of light detected by the detection means, hemoglobin signals dependent on changes in concentration of oxygenated hemoglobin (oxy-Hb) and deoxygenated hemoglobin (deoxy-Hb) in the subject; and a storage section for storing the hemoglobin signals. In the biological light measurement device, the computing section calculates a relative value using a hemoglobin signal at a prescribed measurement channel for the first task and a hemoglobin signal at another prescribed measurement channel for the second task.

### Advantageous Effects of Invention

Using the biological light measurement device of the present invention makes it possible to objectively assess the mood state of a subject in an everyday environment.

Also, configuring the biological light measurement device such that computed results are stored in a storage section makes it possible to assess, based on the stored data, temporal changes in mood state of a subject.

### BRIEF DESCRIPTION OF DRAWINGS

FIG. 1 is a block diagram showing the configuration of a biological light measurement device according to an embodiment of the present invention.

FIG. 2 shows tables stored in the storage section of the biological light measurement device according to an embodiment of the present invention.

FIG. 3 shows an example display displayed in the display section of the biological light measurement device according to an embodiment of the present invention.

FIG. 4 shows example probes each including first and second measurement channels of the biological light measurement device according to an embodiment of the present invention.

FIG. 5 is a block diagram showing an example configuration of the biological light measurement device according to an embodiment of the present invention.

FIG. 6 is a block diagram showing an example configuration of the biological light measurement device according to an embodiment of the present invention.

FIG. 7 is a block diagram showing an example configuration of the biological light measurement device according to an embodiment of the present invention.

FIG. 8 shows an example display displayed in the display section of the biological light measurement device according to an embodiment of the present invention.

FIG. 9 is a schematic diagram showing an example spatial WM task.

FIG. 10 is a schematic diagram showing an example verbal WM task.

FIG. 11 is a diagram showing temporal changes in hemoglobin (Hb) signals obtained by the biological light measurement device according to an embodiment of the present invention.

FIG. 12 (left) shows a correlation between brain activity signals and POMS depression scores on a spatial WM task and FIG. 12 (right) shows a correlation between brain activity signals and POMS depression scores on a verbal WM task.

FIG. 13 shows a 3×10 probe configuration, in which 15 irradiation channels and 15 detection channels are alternately arranged, and measurement channels; and the locations of the measurement channels on the cerebral cortex surface and an approximate arrangement of the DLPFC and frontal pole regions.

FIG. 14 shows an example table which is stored in the storage section of the biological light measurement device according to an embodiment of the present invention and which lists stimulus types for presentation to a subject and the corresponding channels to be measured.

FIG. 15 is a block diagram showing an example configuration of the biological light measurement device according to an embodiment of the present invention.

FIG. 16 is a schematic diagram showing an example verbal WM task.

FIG. 17 is a schematic diagram showing an example verbal WM task.

FIG. 18 shows an example display displayed in the display section of the biological light measurement device according to an embodiment of the present invention.

FIG. 19 shows an example display displayed in the display section of the biological light measurement device according to an embodiment of the present invention.

FIG. 20 shows an example display displayed in the display section of the biological light measurement device according to an embodiment of the present invention.

FIG. 21 shows an example display displayed in the display section of the biological light measurement device according to an embodiment of the present invention.

FIG. 22 shows an example display including a guidance for probe setting displayed in the display section of the biological light measurement device according to an embodiment of the present invention.

FIG. 23 shows an example display including a guidance for probe setting displayed in the display section of the biological light measurement device according to an embodiment of the present invention.

FIG. 24 is a flowchart showing an example procedure for the biological light measurement device, provided with a mood assessment mode, according to an embodiment of the present invention.

FIG. 25 is a flowchart showing an example procedure for the biological light measurement device, provided with a mood assessment mode, according to an embodiment of the present invention.

FIG. 26 shows an example display including a guidance for probe setting displayed in the display section of the biological light measurement device according to an embodiment of the present invention.

FIG. 27 shows example displays for grasping a subjective mood state of a subject displayed in the display section of the biological light measurement device according to an embodiment of the present invention.

FIG. 28 shows equations referred to in describing an embodiment of the present invention.

FIG. 29 shows face icons and weather icons corresponding to mood indexes stored in the storage section of the biological light measurement device according to an embodiment of the present invention.

FIG. 30 shows an example display for selecting first and second tasks on the biological light measurement device, provided with a mood assessment mode, according to an embodiment of the present invention.

### DESCRIPTION OF EMBODIMENTS

Embodiments of the present invention will be described below in detail with reference to drawings. In the following example, mood assessment which cannot be performed by fMRI is performed by biological light measurement in an everyday environment. In summary, mood assessment is performed based on the following new finding that brain activity signals reflecting memorization and memory retention by working memory represent the everyday mood of a healthy individual.

By performing a total of three measurements on a total of four healthy subjects at two-week intervals (the second measurement was performed two weeks after the first measurement, and the third measurement was performed two weeks after the second measurement), the finding based on which the problem can be solved has been obtained. The measurements were performed in the following manner.

### <Biological Light Measurement>

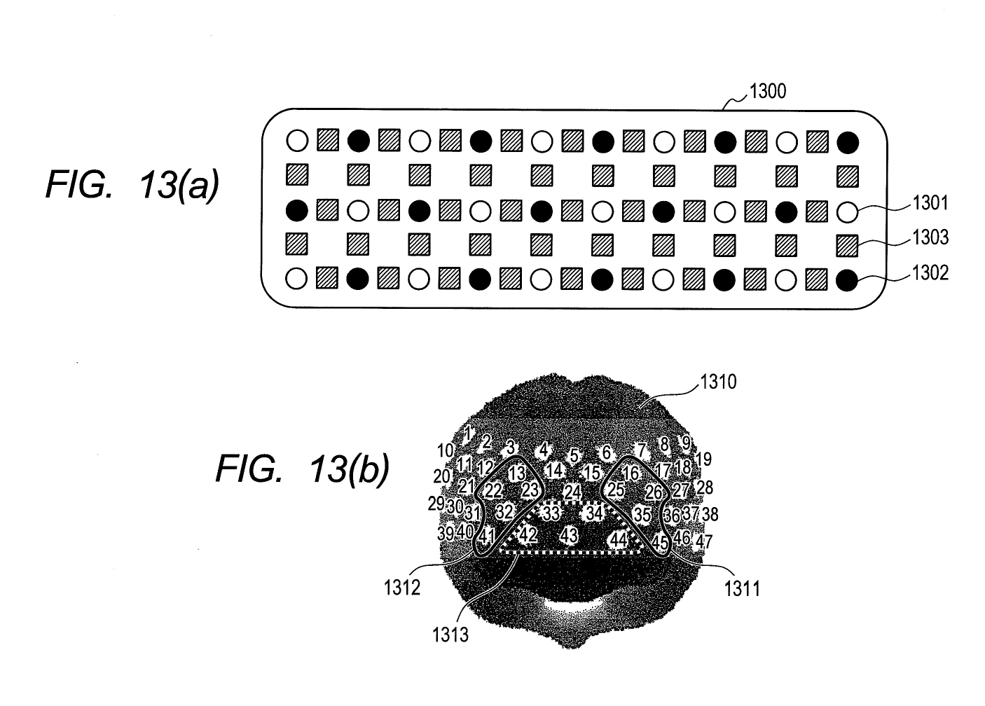

A biological light measuring probe 1300 of a 3×10 configuration in which 15 irradiation channels 1301 and 15 detection channels 1302 are alternately arranged as shown in FIG. 13(a) is attached to the frontal lobe area of each subject and hemoglobin (Hb) signals are obtained as brain activity data from 47 measurement channels (ch). At this time, the measurement channels on the cerebral cortex surface 1310 are located as shown in FIG. 13(b) and are numbered from 1 to 47 representing the corresponding measurement channel numbers. Areas corresponding to the left and right dorsolateral prefrontal cortexes (DLPFC) are enclosed by solid lines 1311 and 1312, respectively, and the region corresponding to the frontal pole around the center of the prefrontal area is enclosed by broken line 1313. Each subject is assigned two types of tasks, i.e. a spatial working memory (WM) task and a verbal working memory task, and the brain activities caused by the respective tasks are assessed.

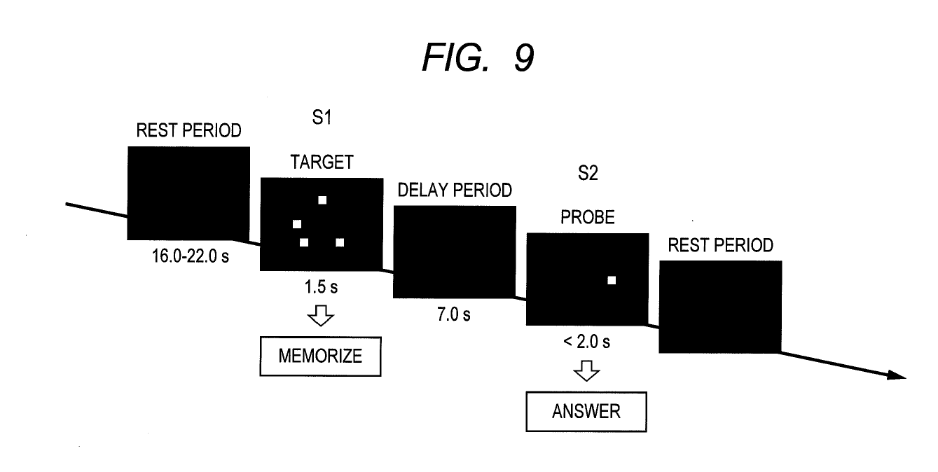

The spatial WM task is schematically illustrated in FIG. 9. An image to be memorized (S1) in which, of the eight squares at eight locations around a central fixation point, those at four or two locations are shown white with the other squares shown gray is presented for 1.5 seconds. Then, seven seconds later, an image (S2) in which, of the eight squares, only one is shown white is presented. Each subject who has been instructed to memorize the locations of the white squares in image S1 is instructed to answer whether the white square shown in image S2 coincides with any of the white squares shown in image S2.

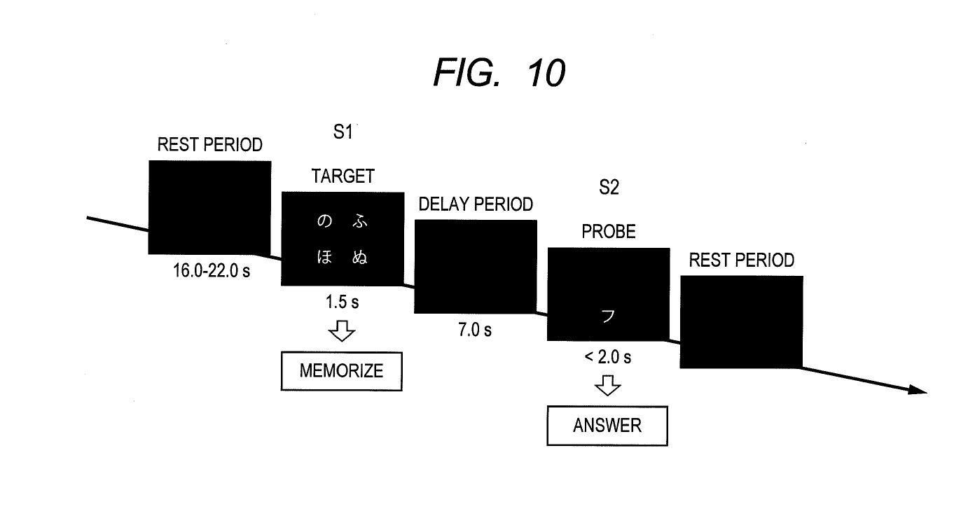

The verbal WM task is schematically illustrated in FIG. 10. An image (S1) in which four or two hiragana characters at four or two locations around a central fixation point are shown is presented for 1.5 seconds. Then, seven seconds later, an image (S2) in which one katakana character is shown is presented. Each subject is instructed to memorize the hiragana characters shown in the first image S1 and to answer whether the katakana character shown in the next image S2 corresponds to any of the memorized hiragana characters. In this task, different kinds of kana characters are shown between images S1 and S2, so that each subject is required to make determination based not on character shape information but on phonological information.

Both the spatial WM task and the verbal WM task require each subject to input a reply by pressing a button of an input means such as a controller or a mouse.

In analysis, an oxygenated Hb signal and a deoxygenated Hb signal are obtained based on time series data measured through each channel for each subject. In each WM task, a period of 8.5 seconds from presentation of the first image (S1) to presentation of the second image (S2) is referred to as a task period, and a period of 25.5 seconds including the task period, one second preceding the task period and 16 seconds following the task period is treated as one block. The data given by each block is baseline-corrected using a line generated by first-order fitting the data obtained from the first one second and last four seconds of each block. It goes without saying that the length of time of each block need not necessarily be the same as described above. Namely, the time length of each task and the lengths of times preceding and following each task to be included in each block may be appropriately changed.

### <Questionnaire>

In order to assess the relationship caused by presentation of the above WM tasks between the state of brain activity of each subject and the mood of each subject, a POMS score reflecting the mood state of each subject during a past week period was obtained using a standardized questionnaire for assessing the mood of each subject “POMS brief form” (“Profile of Mood States—Brief Form, Guide and Case Examples” Kazuhito Yokoyama, Kaneko Shobou, 2005). The questionnaire gives 30 question items, for example, “Tense,” “Lively” and “Sad,” each accompanied by five common selectable answers, i.e. “Not at all,” “Slightly,” “To some extent,” “Considerably” and “Extremely,” and each subject is instructed to select one of the five answers for each question item. Based on the answers given by each subject, a POMS score corresponding to one of six mood state levels, i.e. “Tension-Anxiety,” “Depression-Dejection,” “Anger-Hostility,” “Vigor,” “Fatigue” and “Confusion,” was determined for each subject.

### <Results>

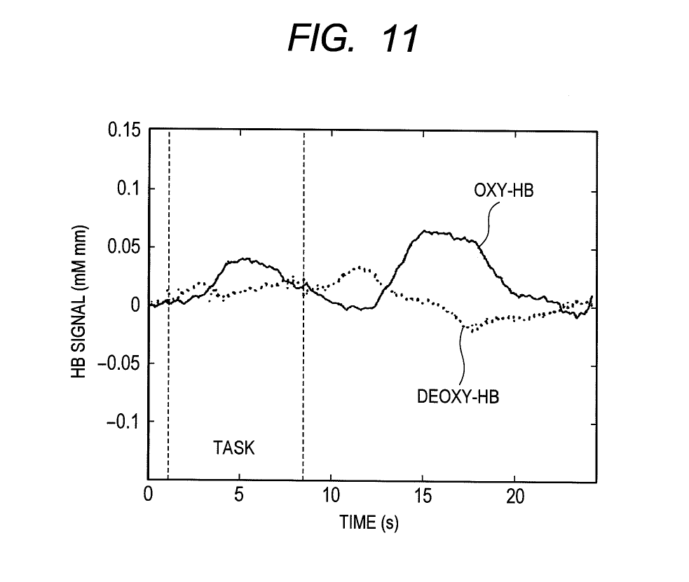

Study of the Hb signals indicated for both the spatial WM task and the verbal WM task that the oxygenated Hb signal locally increased in synchronism with the tasks and that the deoxygenated Hb signal locally decreased in synchronism with the tasks (FIG. 11). The main active parts of the brain of each subject were regions corresponding to the left and right DLPFCs. The DLPFCs are regions including the middle frontal gyrus (Brodmann area 46, BA46) and are known to be activated by a WM task. The spatial characteristics of brain activities were analogous under different task conditions, and no difference attributable to the difference in task type, i.e. the difference between the spatial WM task and the verbal WM task was confirmed. As for temporal changes in the Hb signals in active parts, too, no difference attributable to the difference in task type was observed.

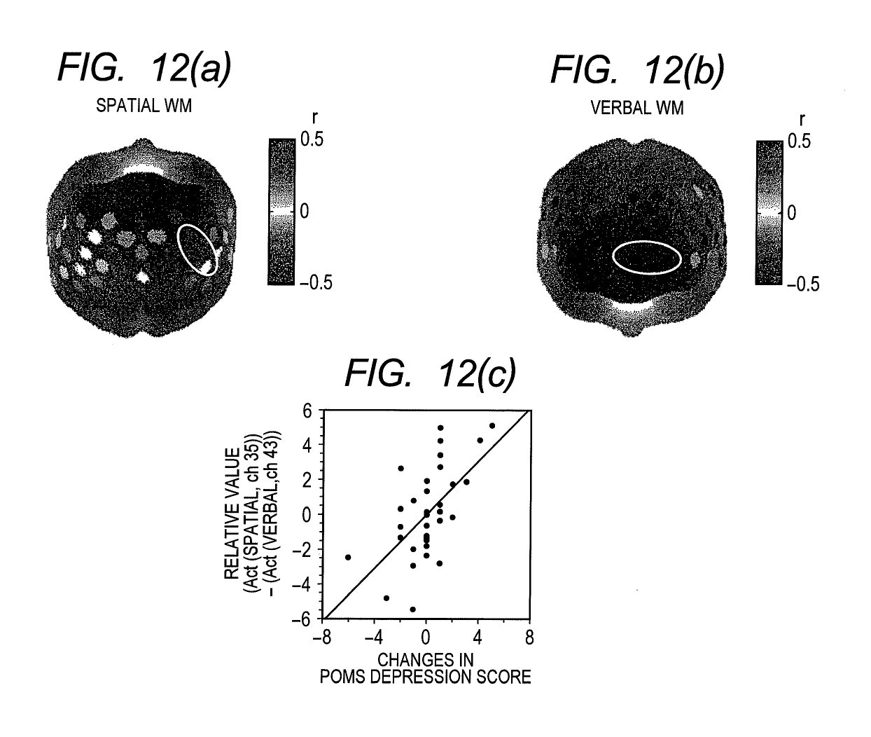

The magnitude of brain activity (Act) was defined as the average value of the oxygenated Hb signal during the period between 5 seconds after the start of presentation of S1 and 8.5 seconds after the start of presentation of S1, and the correlation between Act and the POMS score was studied. As a result, it was found regarding the spatial WM task that, in ch35 and ch45 included in the left DLPFC 1311, there is a positive correlation between the differences in Act between measurements and the differences in POMS depression score between measurements (FIG. 12(a)).

It was also found regarding the verbal WM task that, in ch43 and ch44 in the vicinity of the center of the frontal region coinciding with the frontal pole 1313, there is a negative correlation between the differences in Act between measurements and the differences in POMS depression score between measurements (FIG. 12(b)). Based on the above results, relative values between the differences in Act between measurements on ch35 for the spatial WM task and the differences in Act between measurements on ch43 for the verbal WM task were obtained, and it was found that there is a positive correlation between the relative values thus obtained and changes in POMS depression score (FIG. 12(c)).

A mood state assessment method in which, as described above, the mood state of each subject is assessed by assessing brain activity signals on different tasks at spatially different measurement channels and obtaining their relative value is a new method.

In biological light measurement, they have not compared brain activity signals between different measurement channels. The reason for this is that the Hb signal obtained at each measurement channel being the product (AC-L) of the change in hemoglobin concentration (AC) and the optical path length (L) is dependent not only on Hb concentration changes caused by brain activities but also on the optical path length L. Even though the optical path length L possibly differs between measurement channels, strictly determining such differences is difficult, so that, in related-art cases, Hb signals at different measurement channels are not compared. However, the present inventors have found out that a depression-related index can be obtained by comparing Hb signals at different measurement channels for each of different tasks.

Based on the above finding, concrete configurations of a biological light measurement device for achieving the above effect and corresponding procedures will be described below as embodiments of the present invention.

### First Embodiment

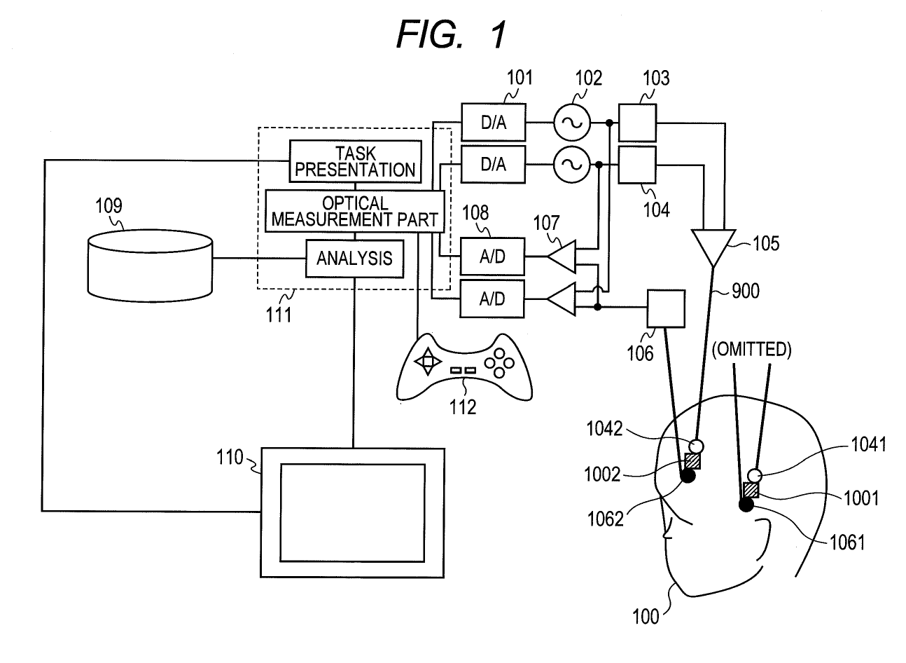

FIG. 1 is a schematic configuration diagram of a biological light measurement device. The biological light measurement device of the present embodiment includes one or plural irradiation means 1041 and 1042 for irradiating a subject with light and one or plural detection means 1061 and 1062 for detecting light transmitted through or reflected from a subject. The irradiation means and detection means are combined to make up plural pairs to be used as plural measurement channels (a first measurement channel 1001 and a second measurement channel 1002). The plural measurement channels are placed at spatially different locations on a subject.

Each of the irradiation means emits light of two wavelengths in a range of about 600 to 900 nm which can be transmitted through a living body. To be more concrete, the irradiation means irradiates a subject 900 with light by putting a light source 103 or 104, which may be a laser diode or an LED, indirect contact with the subject 100 or by putting light from the light source 103 or 104 in contact with the subject 900 using an optical fiber 900. Each of the detection means detects light, similarly to the irradiation means, directly on the subject 100 using, for example, a silicon photodiode, avalanche photodiode or photomultiplier or indirectly by putting the optical fiber 900 in contact with the subject 100 and having light led through the optical fiber 900.

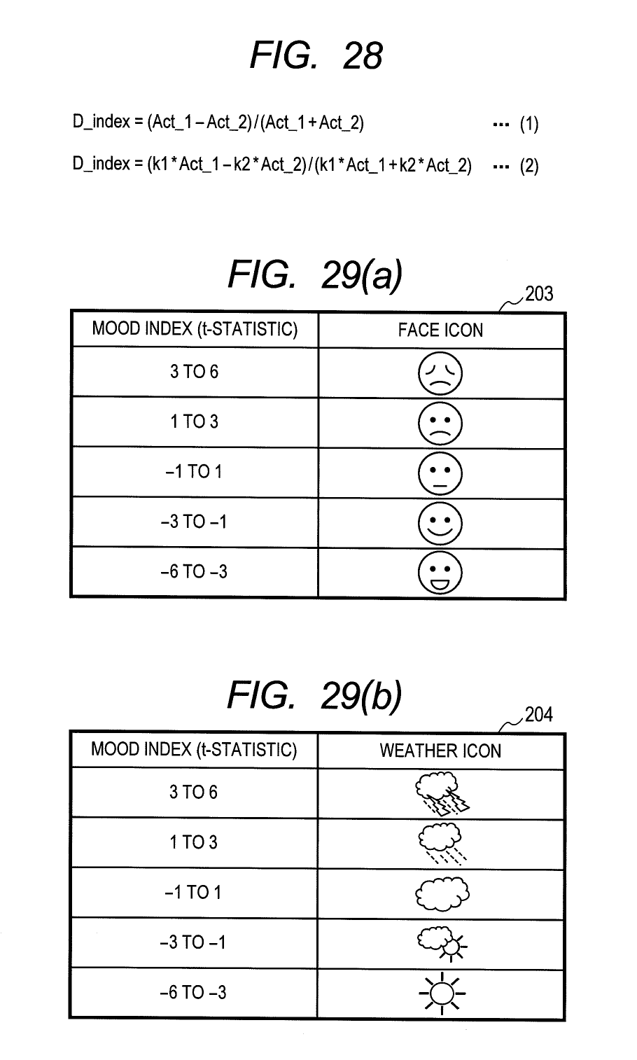

The biological light measurement device has a display section 110 for presenting plural types of tasks (first and second tasks) to the subject 100 and a computing section ill for computing brain activity signals at measurement channels 1001 and 1002. The computing section 111 obtains a brain activity signal at the first measurement channel 1001 of the subject 100 on the first task and a brain activity signal at the second measurement channel 1002 of the subject 100 on the second task. The computing section 111 then calculates a relative value between the respective brain activity signals based on a depression index (D_index) like the one represented by equation (1) shown in FIG. 28.

In equation (1), Act_1 represents a brain activity signal at the first measurement channel 1001 for the first task and Act_2 represents a brain activity signal at the second measurement channel 1002 for the second task.

Each brain activity signal may be weighted as in equation (2) shown in FIG. 28.

The relative value may be calculated as a t-statistic with respect to the difference between Act_1 and Act_2. The above configuration makes it possible to compare brain activity signals at different measurement channels on each of different tasks and obtain an index related with depressed mood of a subject.

### Second Embodiment

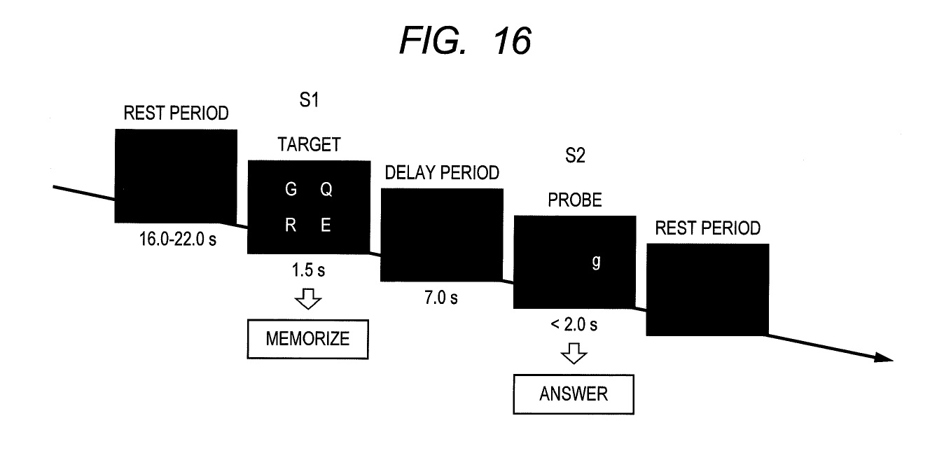

Next, another embodiment of the biological light measurement device according to the present invention will be described. FIG. 16 shows a verbal WM task, unlike the verbal WM task shown in FIG. 10, based on alphabet. In the present embodiment, a subject is instructed to memorize uppercase alphabet letters shown in a first image (S1) and to answer whether or not the lowercase alphabet letter shown in a second image (S2) coincides with any of the letters memorized on S1.

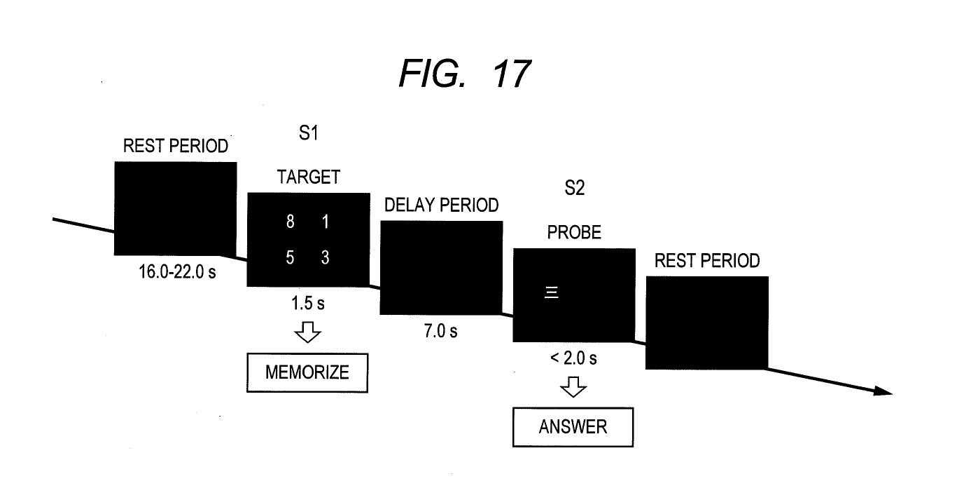

The present embodiment makes it possible to assess, similarly to the first embodiment, the mood of a subject who is more accustomed to alphabet than to Japanese. FIG. 17 shows a verbal WM task, unlike the verbal WM task shown in FIG. 10, based on Arabic numerals and Chinese numerals. A subject is instructed to memorize Arabic numerals shown in a first image (S1) and to answer whether or not the Chinese numeral shown in a second image (S2) coincides with any of the numerals memorized on S1. The present embodiment makes it possible to assess, similarly to the first embodiment, the mood of a subject who is more accustomed to Chinese characters than to Japanese.

### Third Embodiment

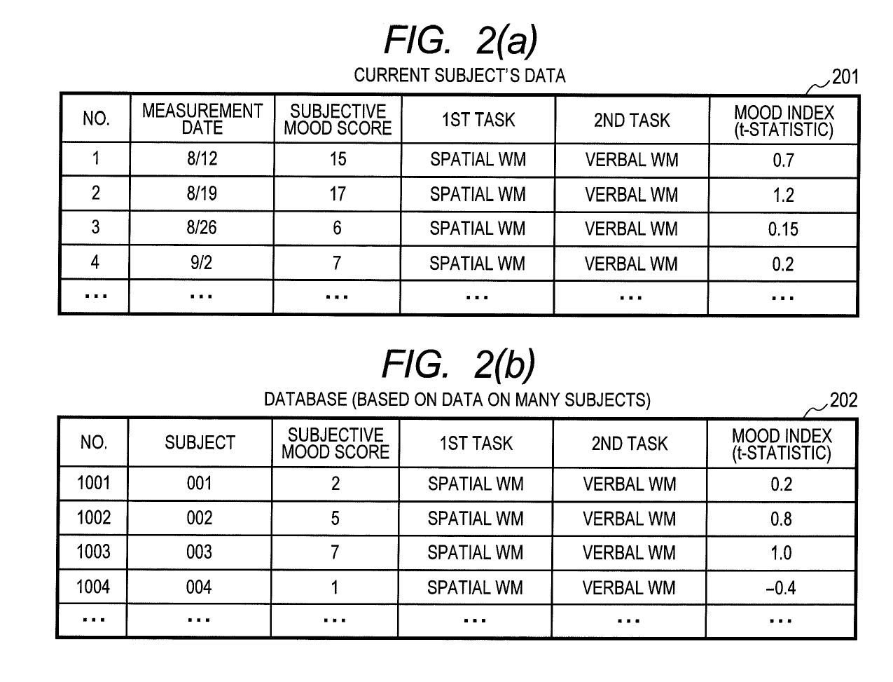

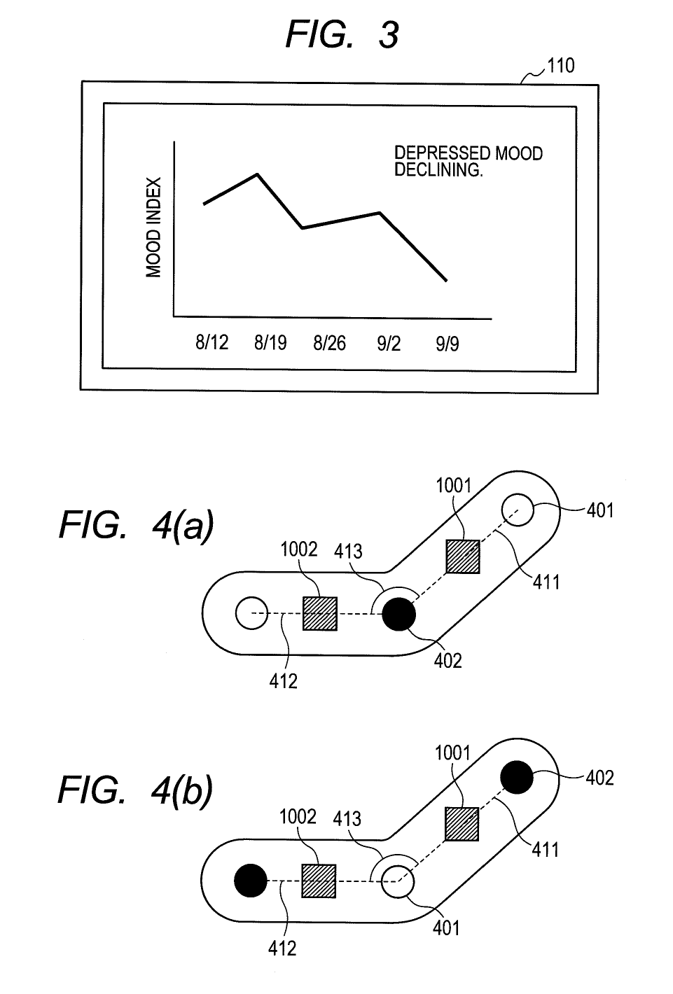

Next, another embodiment of the biological light measurement device according to the present invention will be described. FIG. 2(a) shows a table 201 listing past measurement results on the subject 100. The measurement results include, for each of the measurement dates, a subjective mood score, a first task type, a second task type, and a t-statistic, i.e. a mood index. The measurement results are stored in a storage section 109. When a new mood index is obtained, the computing section 111 adds it to the table 201 to have it stored in the storage section 109. The computing section 111 can then read the past and current mood indexes from the table 201 and display them as a graph in the display section 110 as shown in FIG. 3. Such a graph display enables visual comparison to determine whether the mood state of the subject 100 has improved or deteriorated from the past.

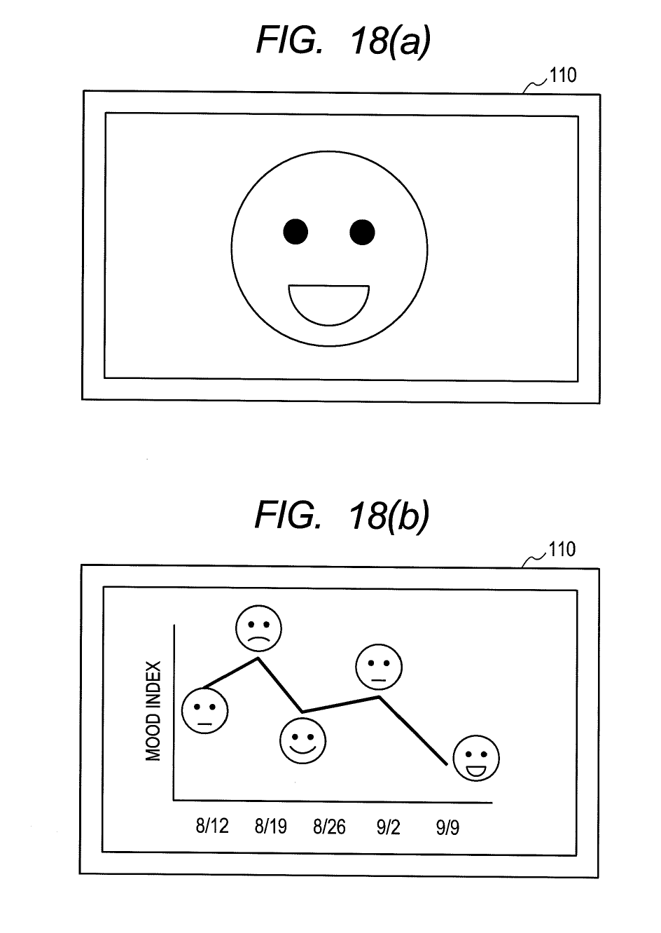

Furthermore, a table of symbols corresponding to mood indexes as shown in FIG. 29 may be stored in the storage section 109, allowing the symbols to be read out to represent the mood indexes obtained using the biological light measurement device. FIG. 29(a) is a table 203 showing mood indexes and corresponding face icons. As shown, a large mood index is represented by a displeased expression and smaller mood indexes are represented by more smiley expressions. When a mood index is obtained as a result of biological light measurement, the computing section 111 can read out the table 203, select the symbol corresponding to the mood index obtained, and display the selected symbol in the display section 110, for example, as shown in FIG. 18(a). It is also possible, when showing a graph of past and current mood states, to read out the table 203 and show the face icons corresponding to the past and current mood states of the subject on a graph so as to represent mood state changes by correspondingly changing face icons, for example, as shown in FIG. 18(b).

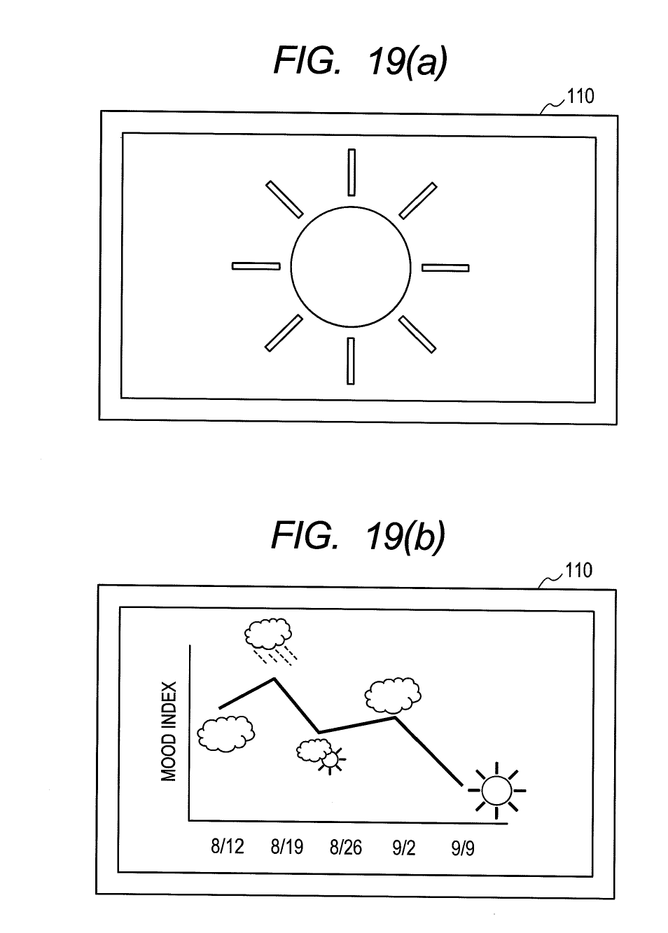

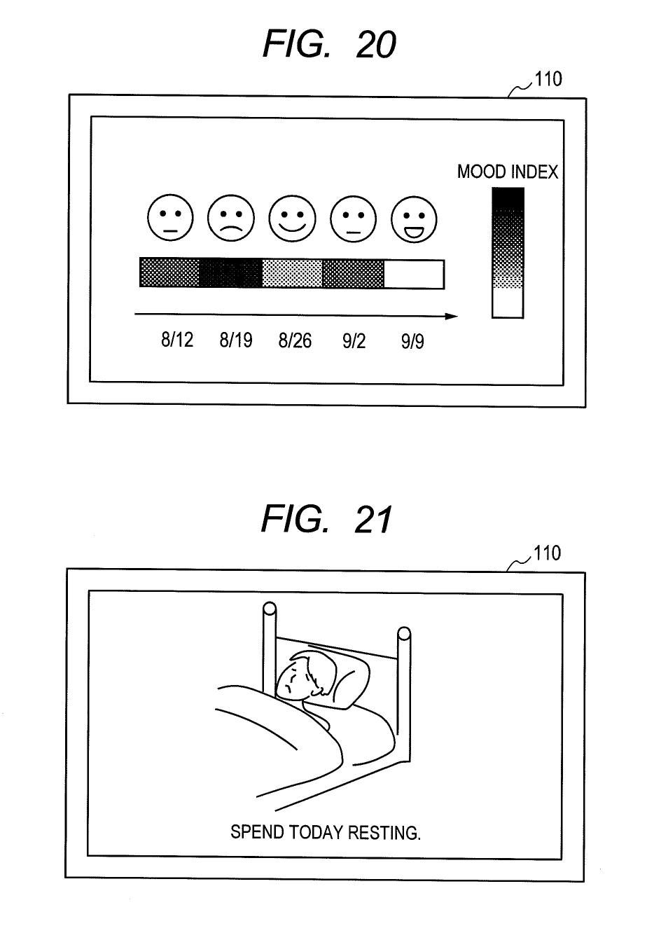

Also, a table 204 containing, as shown in FIG. 29(b), weather icons, instead of the face icons, to represent mood indexes may be stored in the storage section 109 so as to replace the face icons shown in FIG. 18 by the weather icons as shown in FIGS. 19(a) and 19(b). Namely, a small mood index is represented by a sun symbol; a large mood index is represented by a rain symbol; and an intermediate mood index is represented by a cloud symbol. As shown in FIG. 20, mood indexes can also be represented by color shading. When a mood index value is large, a person resting in bed may be displayed as shown in FIG. 21 to recommend a rest.

The above configuration enables visual observation of a subject's current mood state or mood state changes from the past to allow the subject to recognize his or her own mood state.

### Fourth Embodiment

Next, another embodiment of the biological light measurement device according to the present invention will be described. According to the finding described in the beginning of “BEST MODE FOR CARRYING OUT THE INVENTION,” the relative value between the brain activity signal obtained at the measurement channel (ch35 shown in FIG. 13(b)) included in the left DLPFC 1311 on the spatial WM task (the first task) and the brain activity signal obtained at the measurement channel (ch43 shown in FIG. 13(b)) in the vicinity of the center of the frontal region coinciding with the frontal pole 1313 on the verbal WM task (the second task) has a positive correlation with the POMS depression score (FIG. 12(c)). Therefore, in cases where the first task is a spatial WM task for which the first measurement channel is the left DLPFC and where the second task is a verbal WM task for which the second measurement channel is the frontal pole, it is only required to obtain brain activity signals at a minimum of two measurement channels, and it is not necessary to perform measurement over such a wide frontal lobe area as covered by the probe shown in FIG. 13(a).

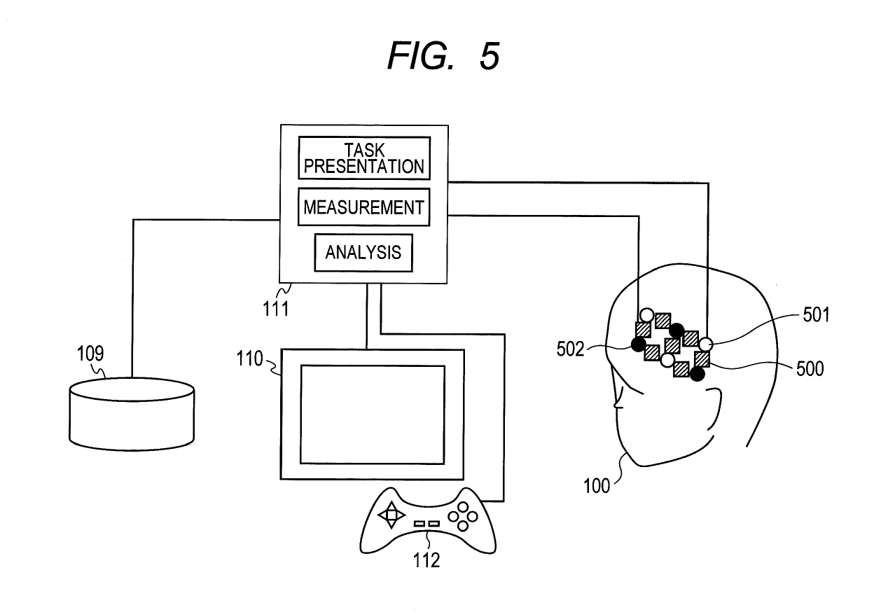

The probe for realizing such two measurement channels can be arranged as shown in FIGS. 4(a) and 4(b). In the probe shown in FIG. 4(a), light emitted from two irradiation channels 401 is detected at a single detection channel 402 thereby realizing a first measurement channel 1001 and a second measurement channel 1002.

In the probe shown in FIG. 4(b), light emitted from one irradiation channel 401 is detected at two detection channels 402 thereby realizing a first measurement channel 1001 and a second measurement channel 1002. In the probes shown in FIGS. 4(a) and 4(b), line 411 connecting the irradiation channel and detection channel that realize the first measurement channel and line 412 connecting the irradiation channel and detection channel that realize the second measurement channel form an angle 413.

Based on the above information, when the location corresponding to ch35 shown in FIG. 13(b) is used as the first measurement channel and the location corresponding to ch43 shown in FIG. 13(b) is used as the second measurement channel, the angle 413 is to be 120 degrees. Since individual subjects have differently shaped heads, the optimum locations of the first and second measurement channels are considered to differ between subjects.

For example, when, for a subject, using the location corresponding to ch35 shown in FIG. 13(b) as the first measurement channel and the location corresponding to ch44 shown in FIG. 13(b) as the second measurement channel, the angle 413 is to be 90 degrees. Also, when, for a subject, using the location corresponding to ch45 shown in FIG. 13(b) as the first measurement channel and the location corresponding to ch44 shown in FIG. 13(b) as the second measurement channel, the angle 413 is to be 180 degrees.

Namely, in the probes shown in FIGS. 4(a) and 4(b) for the present embodiment, when the angle 413 ranges from 90 to 180 degrees, measurement can be performed using a measurement channel included in the left DLPFC 1311 as a first measurement channel and a measurement channel included in the frontal pole 1313 as a second measurement channel. As described above, the probes shown in FIGS. 4(a) and 4(b) of the present embodiment make it possible to perform measurement using the left DLPFC 1311 as a first measurement channel and the frontal pole 1313 as a second measurement channel while also having an effect to reduce the number of irradiation channels and detection channels for realizing the measurement channels.

### Fifth Embodiment

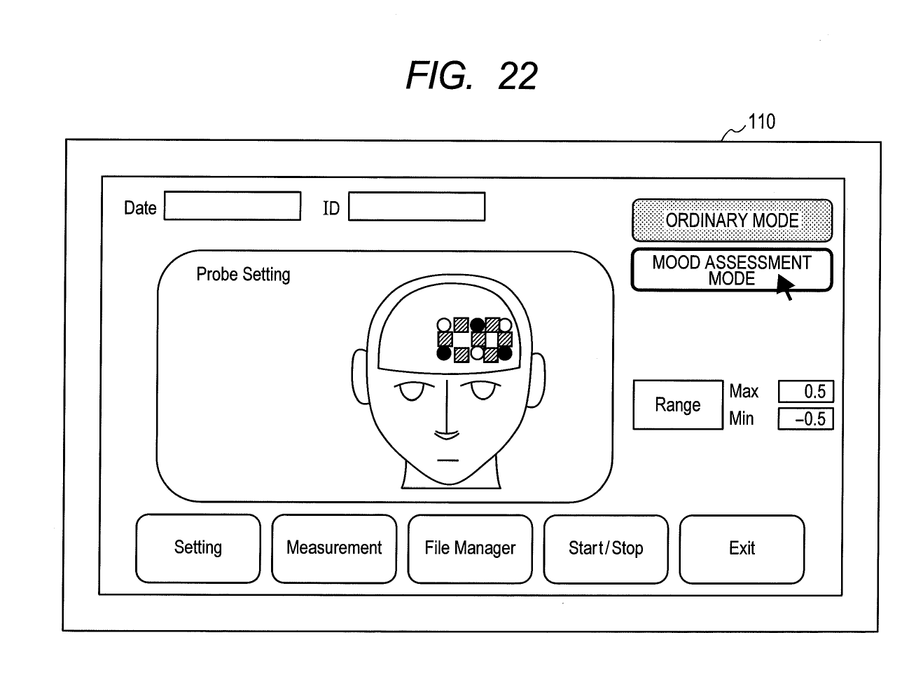

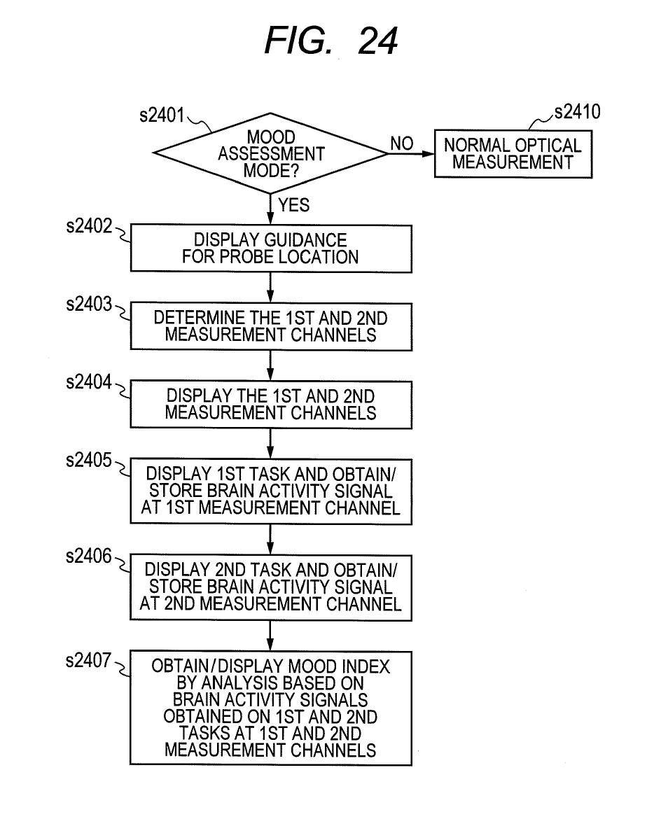

Next, another embodiment of the biological light measurement device according to the present invention will be described. FIG. 5 shows a biological light measurement device which has plural measurement channels 500 with plural irradiation channels 501 and plural detection channels 502 alternately arranged. FIG. 22 shows an example of the display section 110 included in the biological light measurement device provided with “mood assessment mode.” In the present embodiment, the computing section 111 performs processing according to the flowchart shown in FIG. 24.

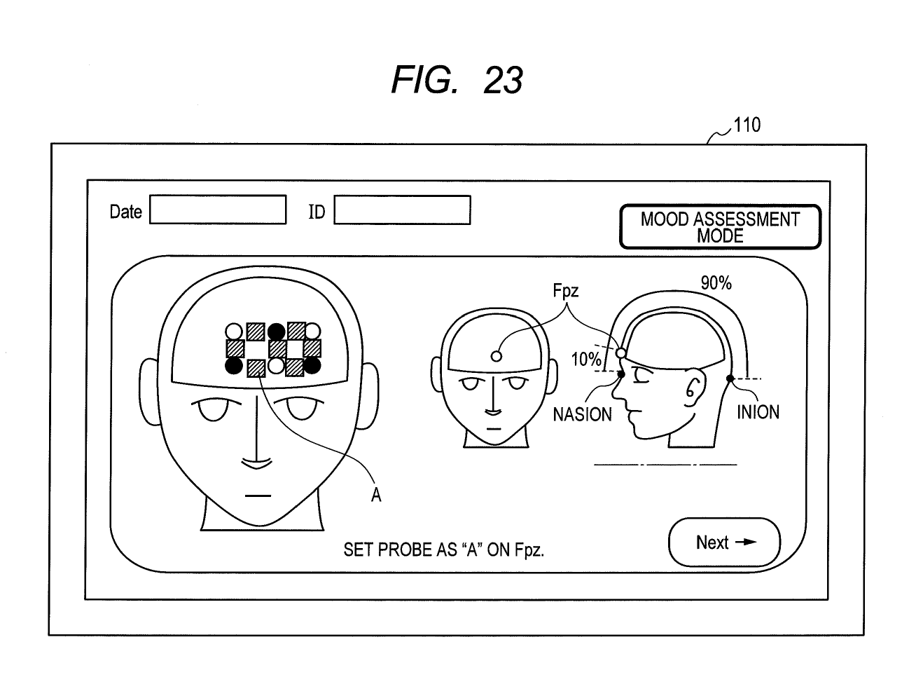

In the biological light measurement device having the mood assessment mode, mode selection buttons for “ordinary mode” and “mood assessment mode” are displayed as shown in FIG. 22 and either mode can be selected using an input means, for example, a controller or a mouse. When the normal mode is selected, namely, when NO is selected in step s2401 shown in FIG. 24, processing advances to step s2410 to perform ordinary biological light measurement. When the mood assessment mode is selected, namely, when YES is selected in step s2401 shown in FIG. 24, processing advances to step s2402 and a guidance message urging the operator to set a probe is displayed in the display section 110 as shown in FIG. 23.

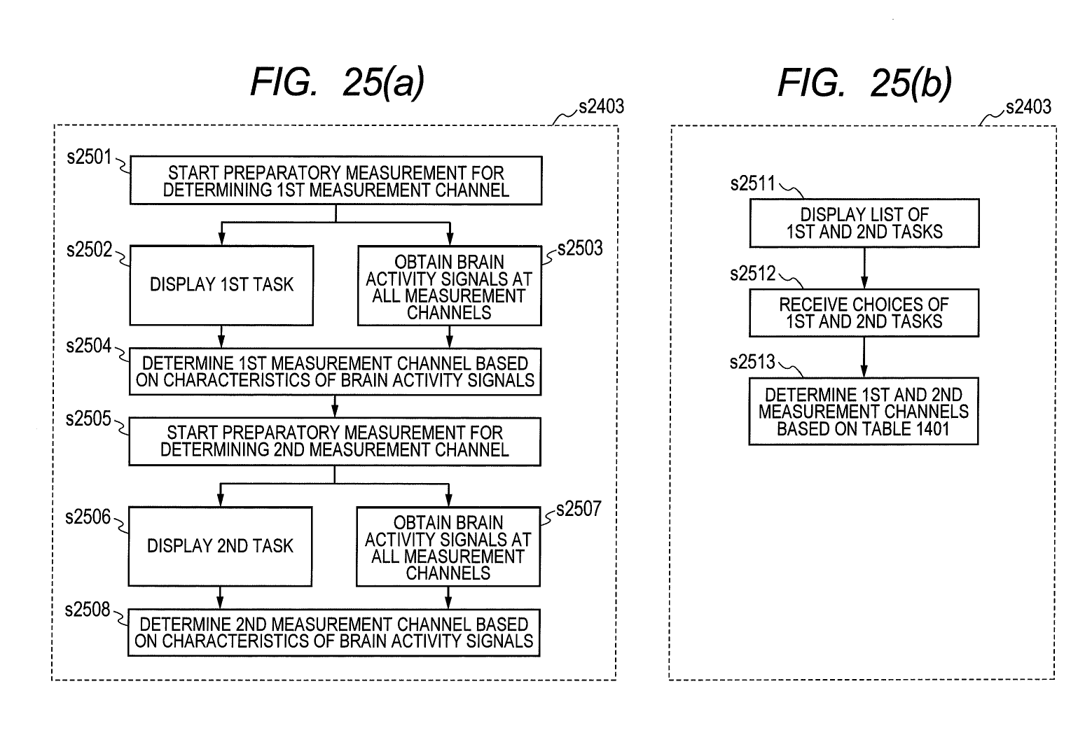

For example, the guidance message displayed in the display section 110 (FIG. 23) urges the operator to set a probe as measurement channel “A” on “Fpz” based on the International 10-20 System. When the probe is set in accordance with the guidance and “Next” button is pressed, processing advances to step s2403 shown in FIG. 24 where the computing section 111 determines a first measurement channel and a second measurement channel. In step s2403, the first and second measurement channels are determined following the flowchart shown in FIG. 25(a). Namely, first in step s2501, preparatory measurement for determining a first measurement channel is started. In step s2502, a first task is displayed in the display section 110 and, in step s2503, brain activity signals at all measurement channels are obtained on the first task. Subsequently, in step s2504, the first measurement channel is determined based on characteristics of the brain activity signals (for example, based on the magnitudes of the brain activity signals).

Next, in step s2505, preparatory measurement for determining a second measurement channel is started. In step s2506, a second task is displayed in the display section 110 and, in step s2507, brain activity signals at all measurement channels are obtained on the second task. Subsequently, in step s2508, the second measurement channel is determined based on characteristics of the brain activity signals.

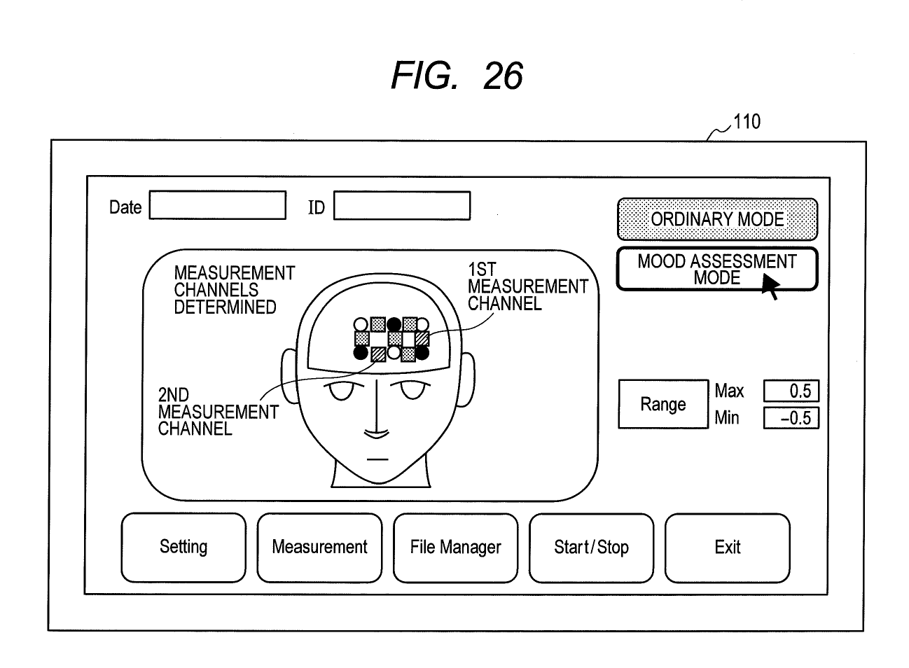

When step s2403 shown in FIG. 24 is finished following the flowchart shown in FIG. 25(a), processing advances to step 52404 where determination results are displayed as shown in FIG. 26. Subsequently, in step s2405, the brain activity signal at the first measurement channel is obtained on the first task. At this time, acquisition of a brain activity signal is required only at the first measurement channel, so that it is not necessary to use any irradiation channel or detection channel not related with the first measurement channel. Also, in step s2406, the brain activity signal at the second measurement channel is obtained on the second task. At this time, similarly to the above case of the first measurement channel, acquisition of a brain activity signal is required only at the second measurement channel. Based on the results of brain activity signal acquisition in steps s2405 and s2406, a mood index is calculated in step s2407 and the calculated result is displayed in the display section 110.

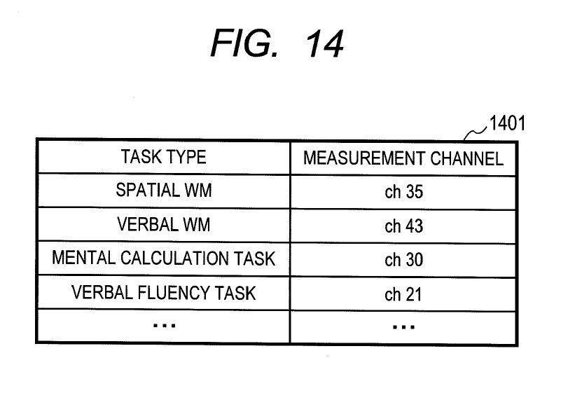

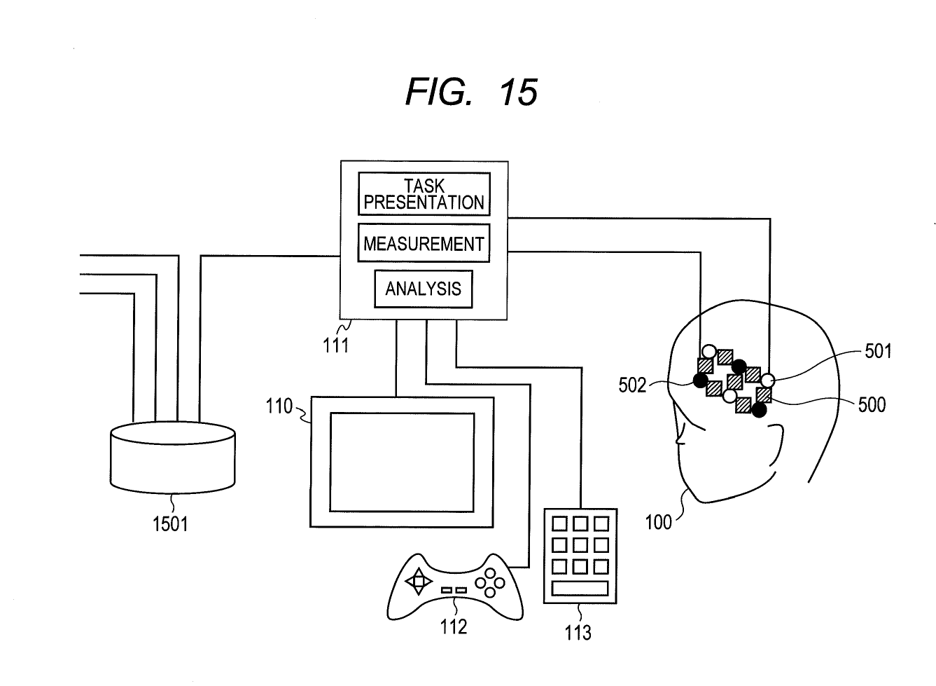

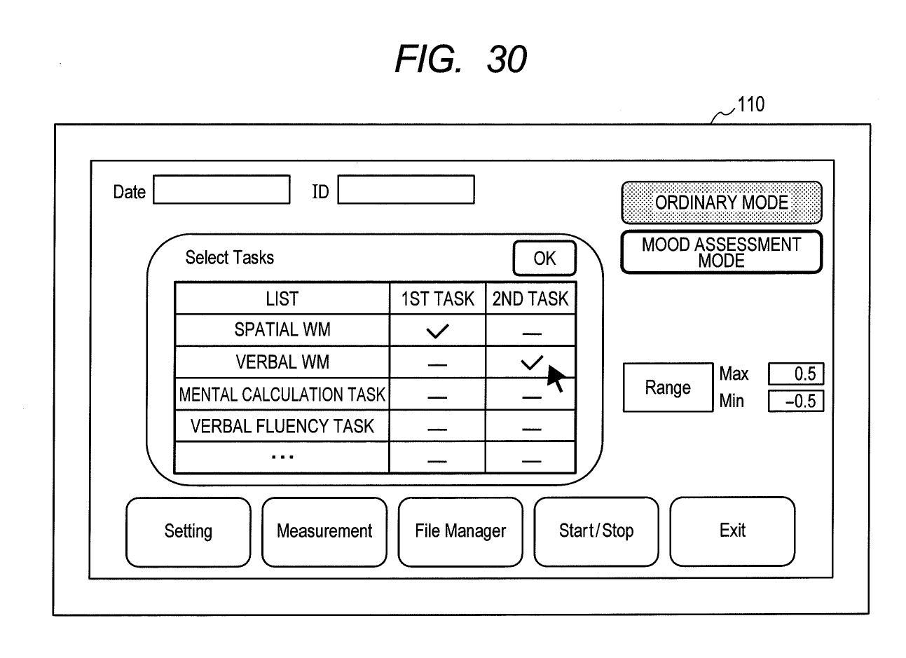

Step s2403, shown in FIG. 24, for determining the first and second measurement channels may also be performed as indicated in FIG. 25(b). With plural types of tasks stored in the storage section 109 beforehand, they are, in step s2511, displayed as a list in the display section 110 as shown in FIG. 30. The list includes check boxes allowing a first task and a second task to be selected from the listed tasks. After a task is selected as a first task and another task is selected as a second task using the input means 112 in step s2512, pressing the “OK” button shown in FIG. 30 causes the computing section 111 to receive the task choices. The storage section 109 stores a table 1401 which lists, as shown in FIG. 14, task types and corresponding measurement channels. In step s2513, the computing section 111 reads out the table 1401 and determines the measurement channels corresponding to the first and second tasks selected in step s2512.

According to the present embodiment, a biological light measurement device having many measurement channels can receive choices of a mood assessment mode and obtain a brain activity signal using only measurement channels required for mood assessment without involving any irradiation channel or detection channel not required for the brain activity signal acquisition. This can achieve a cost reduction, for example, a reduction in power consumption.

### Sixth Embodiment

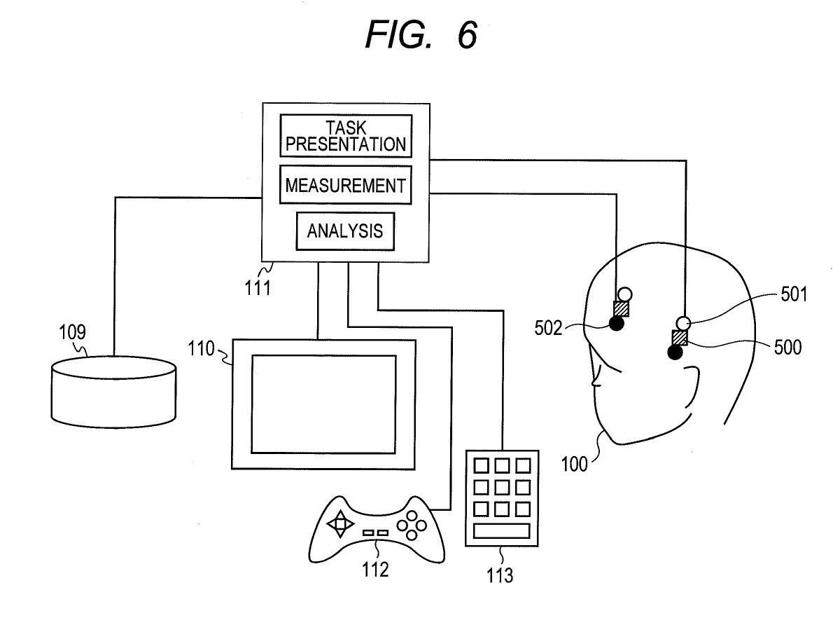

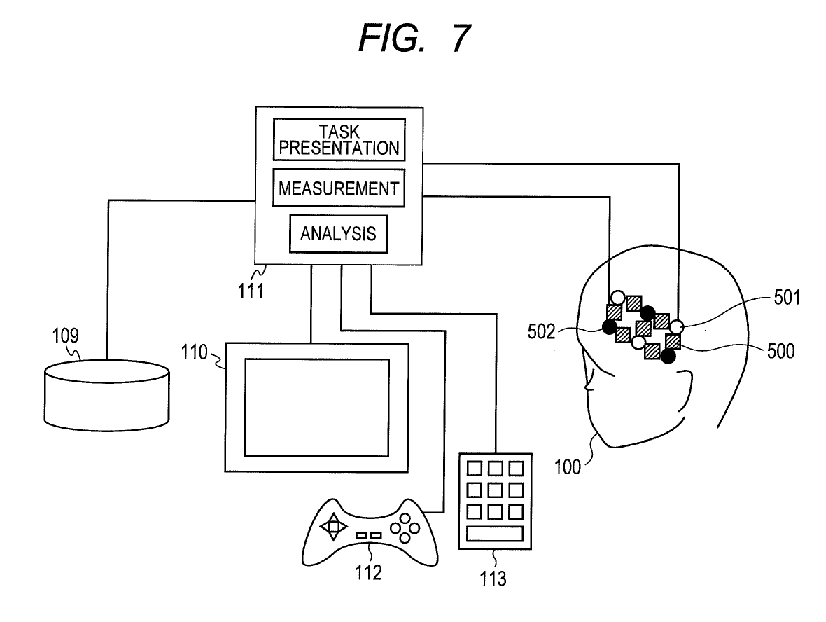

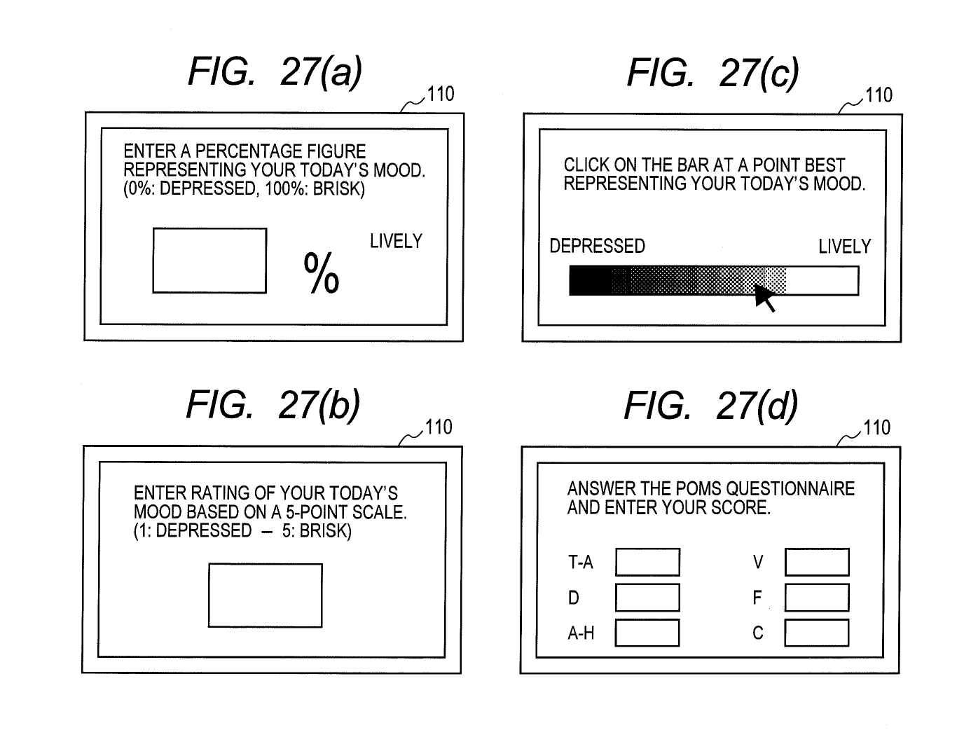

Next, another embodiment of the biological light measurement device according, to the present invention will be described. FIGS. 6 and 7 show a biological light measurement device additionally provided with a mood acquisition means 113. The mood acquisition means is for obtaining a subjective mood state of a subject. The subjective mood state is obtained by having the subject respond to a display such as those shown in FIGS. 27(a) to 27(d) displayed in the display section.

FIG. 27(a) is a display for having a subject enter a figure representing his/her subjective mood state on a percentage basis with 100% representing his/her best subjective mood state. FIG. 27(b) is a display for having a subject rate his/her subjective mood state based on a 5-point scale. FIG. 27(c) is a display for having a subject indicate his/her subjective mood state by a visual analog scale (VAS) method. In this method, a numerical value representing the mood state of a subject can be obtained. For example, the numerical value is 100 when the right end of the bar is clicked and 0 when the left end of the bar is clicked. FIG. 27(d) is a display for instructing a subject to answer the POMS questionnaire and obtaining the mood state of the subject by accepting the resultant input by the subject.

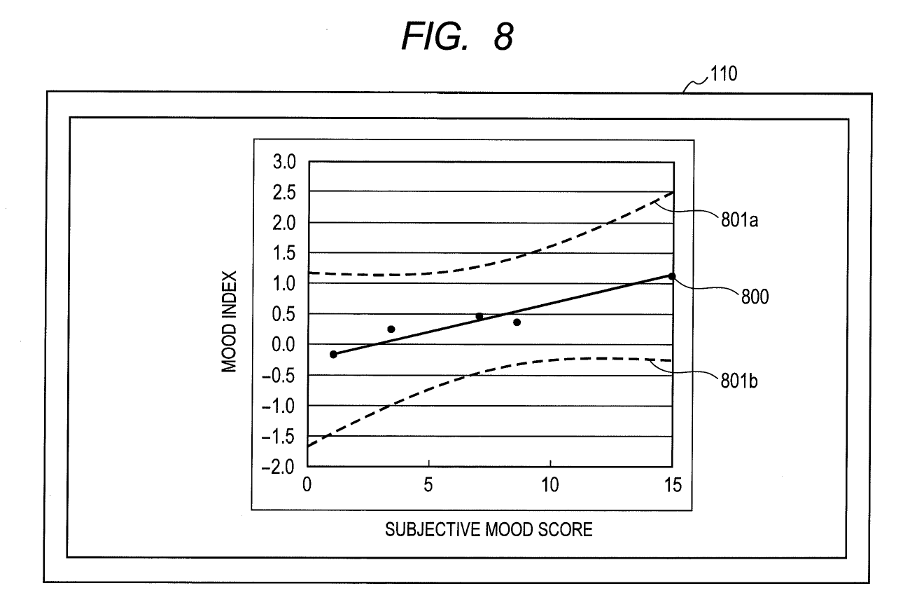

In the present embodiment, the storage section 109 stores accumulated mood index data, like the table 201 shown in FIG. 2(a), obtained based on the past subjective mood states and brain activity signals of the current subject. Corresponding mood index data obtained from the subjective mood states and brain activity signals of many subjects is also stored like the table 202 shown in FIG. 2(b). The computing section 111 reads out the table 202 from the storage section 109 and calculates the 95% confidence interval of the data. Subsequently, the computing section 111 reads out the table 201 containing the current subject's data from the storage section 109 and displays the data as a graph like data channel 800 shown in FIG. 8 while also displaying broken-line curves 801a and 801b representing the 95% confidence interval of the table 201. The present embodiment enables visual comparison to see how the subjective mood of a current subject compares with the corresponding data on many subjects. Namely, the subject can realize the objective level of his/her subjective mood state.

In the present embodiment, a database center 1501 connected via a network may be provided to store mood indexes obtained from subjective mood states and brain activity signals of many subjects. Storing such data in the database center 1501 makes it possible to accumulate the latest data so that the table 202 can be updated to the latest state.

### LIST OF REFERENCE SIGNS

100 Subject
1001 First measurement channel
1002 Second measurement channel
101 Digital-analog converter
102 Modulator
103, 104 Light source
1041, 1042 Irradiation channel
105 Photomixer
106 Detector
1061, 1062 Detection channel
107 Lock-in amplifier
108 Analog-digital converter
109 Storage section
110 Display section
111 Computing section
112 Input means
113 Mood acquisition means
201 Table listing task types and corresponding mood index obtained on each measurement date
202 Table listing task types and corresponding mood index of each of many subjects
203 Table showing face icons corresponding to mood indexes
204 Table showing weather icons corresponding to mood indexes
401 Irradiation channel
402 Detection channel
411 Line connecting a irradiation channel and a detection channel forming a first measurement channel
412 Line connecting a irradiation channel and a detection channel forming a second, measurement channel
413 Angle formed by line 411 and line 412
500 Measurement channel
501 Irradiation channel
502 Detection channel
800 Data representing correspondence between subjective mood scores and mood indexes obtained from brain activity signals of a current subject
801a Broken line indicating an upper boundary of a 95% confidence interval computed based on subjective mood scores and mood indexes obtained from brain activity signals of many subjects
801b Broken line indicating a lower boundary of a 95% confidence interval computed based on subjective mood scores and mood indexes obtained from brain activity signals of many subjects
900 Optical fiber
1301 Irradiation channel
1302 Detection channel
1303 Measurement channel
1310 Cerebral cortex surface as seen from front
1311 Solid line indicating left DLPFC region
1312 Solid line indicating right DLPFC region
1313 Broken line indicating frontal pole region
1401 Table listing task types and corresponding measurement channels
1501 Database center

1. A biological light measurement device, comprising one or a plurality of light emitters for emitting light to a subject; one or a plurality of light detectors for detecting light transmitted through or reflected from the subject; a plurality of measurement channels including a plurality of combinations of the irradiation means and the detection means; a task presentation section which at least presents a plurality of different tasks including a first task and a second task to the subject; a computing section which calculates, based on intensities of light detected in at least two of the measurement channels, hemoglobin signals dependent on changes in concentration of oxygenated hemoglobin and deoxygenated hemoglobin in the subject; and a storage section for storing the hemoglobin signals; wherein the computing section calculates a relative value using a hemoglobin signal of a prescribed measurement channel for the first task and a hemoglobin signal of another prescribed measurement channel for the second task.

2. The biological light measurement device according to claim 1, wherein:
the storage section can store the calculated relative value; and
a display section for displaying the relative value and a past relative value is provided.

3. The biological light measurement device according to claim 1, wherein:
the first task is a spatial WM task; and
the second task is a verbal WM task.

4. The biological light measurement device according to claim 1, wherein:
the relative value is calculated using the following equation:
D_index=(Act_{—}1−Act_{—}2)/(Act_{—}1+Act_{—}2).

5. The biological light measurement device according to claim 2, wherein:
the display section can display a screen for having a mood of a subject entered.
